(19) United States
(12) Patent Application Publication
(10) Pub. No.: US 2026/0338796 A1
(43) Pub. Date: Oct. 29, 2026

(54) PANIC DISORDER

(71) Applicants: University of Helsinki, Helsinki (FI); Max-Planck-Gesellschaft Zur Foerderung Der Wissenschaften E.V., Munich (DE)

(72) Inventors: Iiris Hovatta, Espoo (FI); Angelika Erhardt, Munich (DE); Christoph W. Turck, Munich (DE)

(73) Assignees: University of Helsinki, Helsinki (FI); Max-Planck-Gesellschaft Zur Foerderung Der Wissenschaften E.V., Munich (DE)

(21) Appl. No.: 19/109,077

(22) PCT Filed: Sep. 5, 2023
(86) PCT No.: PCT/EP2023/074294

§ 371 Date: Mar. 5, 2025

(30) Foreign Application Priority Data
Sep. 8, 2022 (GB) 2213124.7

(51) Int. Cl. G01N 33/68

(52) U.S. Cl. CPC G01N 33/6893; G01N 2333/705; G01N 2800/301

(57) ABSTRACT

The invention concerns a method for determining the existence of or susceptibility to panic based upon the presence of serum sortilin related VPS10 domain containing receptor 3 (SORCS3); a kit for performing the afore method(s); at least one antibody and/or at least on probe for use in said afore method or kit; and a treatment regimen for treating panic involving the use of the afore method or kit or antibody or probe.

### FIELD OF THE INVENTION

The invention concerns a method for determining the existence of or susceptibility to panic based upon the presence of serum sortilin related VPS10 domain containing receptor 3 (SORCS3); a kit for performing the afore method; at least one antibody and/or at least one probe for use in said afore method or kit; and a treatment regimen for treating panic involving the use of the afore method or kit or antibody or probe.

### BACKGROUND OF THE INVENTION

A number of psychiatric disorders include an element of panic. Indeed, the term “anxiety spectrum disorder” refers to generalised anxiety disorder (GAD), obsessive-compulsive disorder (OCD), panic disorder (PD), post-traumatic stress disorder (PTSD), social anxiety disorder (also called social phobia), and specific phobias. The invention applies to any one or all of these disorders, providing the patient is susceptible to, or known to, panic. Within PD there are subdivisions such as the panic-agoraphobic spectrum which is characterised by eight clinical features: 1) separation sensitivity, 2) panic-like symptoms, 3) stress sensitivity, 4) medication and substance sensitivity, 5) anxious expectation, 6) agoraphobia, 7) illness phobia and hypochondriasis, 8) reassurance orientation.

Panic is defined by recurrent, unexpected panic attacks, wherein at least one panic attack must be followed by at least one month of persistent concern about having more attacks, worry about the consequences of the attacks, or maladaptive behaviour (e.g., avoidance of work or school activities) related to the attacks. Panic is characterised by one or more of the following symptoms: pounding or racing heart, sweating, chills, trembling, difficulty breathing, weakness or dizziness, tingly or numb hands and chest pain.

The lifetime prevalence of Panic Disorder is 1.7-3.7% and it is twice as common in females than in males. The age of onset is often in early adulthood, and about 80% of cases have a comorbid mental disorder, most often another anxiety or a mood disorder. Like in most psychiatric disorders, the aetiology is complex, consisting of multiple genetic and environmental risk factors.

A meta-analysis of twin studies has estimated Panic Disorder heritability to be ~43%. Panic Disorder has a shared genetic component with other anxiety disorders, and different anxiety disorders often aggregate within individuals and families. Therefore, most previous genome-wide association analyses (GWAS) have studied Panic Disorder as part of a larger composite phenotype of anxiety disorders. Of the Panic Disorder specific GWAS, an early study in 200 Japanese cases and 200 controls identified two significant loci, within the PKP1 (p-value=4.6×10⁻⁰⁸) and TMEM16B (p-value=3.7×10⁻⁹)¹². SNPs within TMEM132D were associated with Panic Disorder (p-value=5.1×10E⁻⁰⁷) in a German study of 216 cases and 222 controls¹³, and this finding was replicated in a multi-ethnic sample of 1670 cases and 2266 controls and validated in a mouse model^{13,14}. The largest GWAS meta-analysis of Panic Disorder to date, with 2,147 cases and 7,760 controls of European origin, did not detect genome-wide significant associations¹⁵.

To examine the genetic architecture of Panic Disorder, we took advantage of the large Finnish register-based sample from the FinnGen (http://finngen.fi), which combines healthcare register data with genetic information, and conducted a GWAS. Since Panic Disorder is more common in females than males, it has been postulated that males may have a higher genetic liability threshold for Panic Disorder, and therefore we also carried out a sex-stratified GWAS.

We then sought to replicate our finding in other populations, using summary level data from the UK Biobank and Estonian Biobank. Finally, we examined serum protein levels of SORCS3, which was our most significant finding from the FinnGen GWAS. Surprisingly, we found this serum protein to be a significant biomarker for panic and so we disclose herein a diagnostic assay and treatment regimen based upon this fact.

### STATEMENTS OF THE INVENTION

According to a first aspect of the invention there is provided a method for determining the existence of, or susceptibility to, panic in an individual to be tested comprising:
(a) providing a sample from the individual to be tested;
(b) detecting, or quantifying the amount of sortilin related VPS10 domain containing receptor 3 (SORCS3), or a part thereof, in said sample; and
(c) where SORCS3 is present at a level above that in healthy individuals, concluding the individual is at an increased risk of developing or exhibiting panic.

In a preferred embodiment of the invention said sample is selected from one or more of the following samples: urine, seminal fluid, blood, blood plasma, lymph fluid, CSF, saliva, sputum, lavage, bronchoaveolar lavage, cerebrospinal fluid and extracellular vesicles. Preferably said sample is serum.

In a further preferred embodiment said method is of relevance in relation to identifying and treating a range of psychiatric disorders that include an element of panic such as, e.g., generalised anxiety disorder (GAD), obsessive-compulsive disorder (OCD), panic disorder (PD), PD with agoraphobia, post-traumatic stress disorder (PTSD), social anxiety disorder (also called social phobia), specific phobias and panic within other psychiatric disorders. The invention also has relevance in relation to identifying and treating psychiatric-related disorders such as those that include panic arising from a primary medical condition, such as COPD, and heart disease.

Reference herein to panic, in particular Panic Disorder or Panic Disorder with Agoraphobia, refers to a disorder characterised by recurrent, unexpected panic attacks, wherein at least one panic attack must be followed by at least one month of persistent concern about having more attacks, worry about the consequences of the attacks, or maladaptive behaviour (e.g., avoidance of work or school activities) related to the attacks. An individual having or suspected of a Panic Disorder is typically diagnosed by conventional means known in the field such as, but not limited to, Panic Disorder Severity Scale (PDSS), Diagnostic and Statistical Manual of Mental Disorders (DSM; latest edition: DSM-5-TR, or according to the ICD-10 (the 10^{th} revision of the International Statistical Classification of Diseases and Related Health Problems (ICD)) definition of panic disorder.

Reference herein to SORCS3 is reference to a type-I receptor transmembrane protein that is a member of the vacuolar protein sorting 10 (VPS10P) receptor family, a group of sorting and signalling receptors central to many pathways in control of neuronal viability and function. SORCS3 is highly expressed in the CA1 region of the hippocampus. Proteins of this family are defined by a vacuolar protein sorting 10 domain at the N-terminus. The N-terminal segment of this domain has a consensus motif for proprotein convertase processing, and the C-terminal segment of this domain is typically characterised by ten conserved cysteine residues. The vacuolar protein sorting 10 domain is typically followed by a leucine-rich segment, a transmembrane domain, and a short C-terminal cytoplasmic domain that interacts with adaptor molecules. The extracellular domain of SORCS3 protein can be shed through regulated proteolytic cleavage and detected in peripheral specimens as herein described. Accordingly, the invention relates to SORCS3 protein/polypeptide or a part thereof, in particular the extracellular domain thereof. In addition, the invention relates to the detection of mRNA of SORCS3/SORCS3 in said sample.

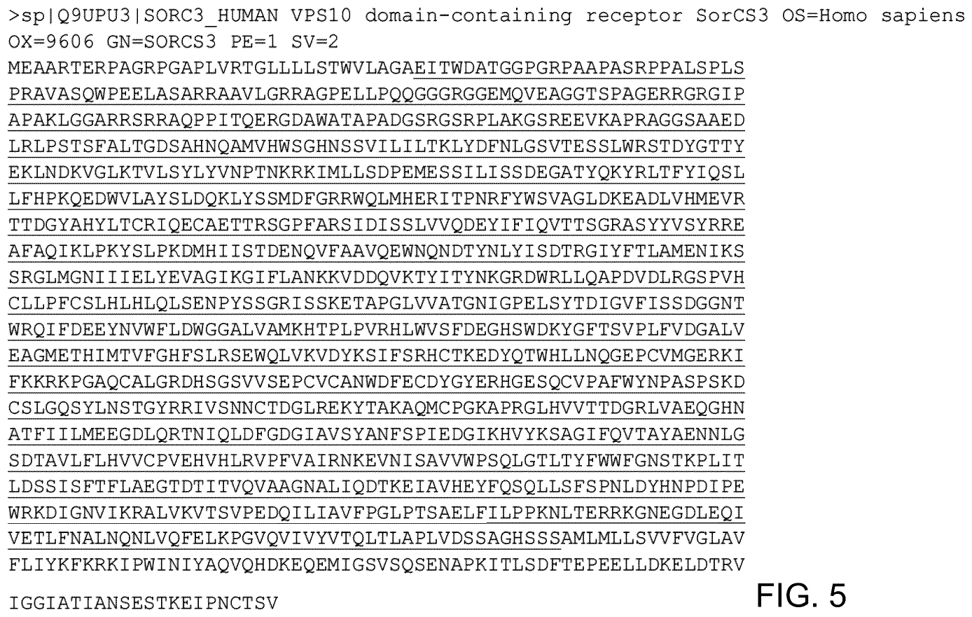

The amino acid sequence structure of this protein is shown in FIG. 5. The extracellular domain is marked by underscoring and contains amino acids 34-1125.

As will be appreciated by those skilled in the art, detection of SORCS3 can be determined by numerous means including, but not limited to, detection of SORCS3 protein, or fragments thereof, specifically the extracellular domain of same, or the detection of a transcript of the gene encoding SORCS3 such as mRNA or cDNA pertaining to same.

Typically, conventional assays are employed for the detection of SORCS3 protein, or fragments thereof specifically the extracellular domain of same, or the detection of a transcript of the gene encoding SORCS3 such as mRNA pertaining to same. These assays are known to those of skill in the art and examples are mentioned below.

In a preferred method of the invention said method is an in vitro assay for the detection or quantification of human SORCS3 protein/polypeptide, or a part thereof, or a transcript of the gene encoding same, in said sample. Preferably, said assay is an immunoassay.

In a preferred embodiment of the invention said method is an enzyme-based assay, ideally an in vitro assay, for the detection or quantification of human SORCS3, or a part thereof, in said sample.

In a preferred method of the invention said method is an enzyme-linked immunosorbent assay (ELISA) using a solid-phase enzyme immunoassay (EIA) to detect the presence of SORCS3, or a part thereof, in a sample using antibodies directed against SORCS3 or part thereof, i.e., the protein to be measured. Typically, the antibodies are specific for at least the extracellular domain of SORCS3. ELISA has been used as a diagnostic tool in medicine for some time and so its features will be known to those skilled in the art.

In the most simple form of an ELISA, SORCS3, or a part thereof from said sample to be tested, are attached to a surface. Then, an antibody specific for SORCS3, or a part thereof, is applied over the surface so it can bind the SORCS3 target. This antibody is linked to an enzyme and after any unbound antibodies are removed, a substance containing the enzyme's substrate is added. If there is antibody-SORCS3 binding, the subsequent reaction produces a detectable signal, most commonly a colour change. However, in some instances the enzyme can be used to catalyse a light emitting reaction such as a fluorescent reaction. Additionally, the ELISA may be sandwiched or layered whereby multiple interacting species are used to detect the target protein/polypeptide and signal the presence of same.

In a preferred method of the invention said method is an immunofluorescence-based assay, ideally an in vitro assay, for the detection or quantification of human SORCS3, or a part thereof, or a transcript of the gene encoding same in said sample.

In a preferred method of the invention said sample is contacted with one or more probes wherein said probe[s] is specific for, and so detects, SORCS3, or a part thereof, or a transcript of the gene encoding same, in said sample. Ideally, said probe is provided with a signalling agent that emits a signal whereby, following typically a washing step to remove unbound probe, the bound probe emits a signal indicative of the presence or amount of said SORCS3, or a part thereof, in said sample.

In a further preferred method of the invention said sample is contacted with one or more further probe[s] wherein said further probe[s] detects SORCS3, or a part thereof, or a transcript of the gene encoding same, and/or an earlier used probe.

In a preferred method of the invention said sample is contacted with a yet further probe wherein said probe detects all or part of SORCS3, or a transcript of the gene encoding same.

In a further preferred method of the invention said SORCS3, or a part thereof, or a transcript of the gene encoding same is detected using mass spectrometry.

Preferably, said SORCS3 amino acid sequence comprises SEQ ID NO: 1 the full protein sequence or SEQ ID NO: 2 the cleavable extracellular domain and shown in FIG. 5 is detected by a method as herein disclosed.

Additionally, or alternatively, in a preferred method of the invention said detecting or quantifying is undertaken by assessing the binding of said antibody or probe to SORCS3 or a part thereof.

In a preferred method of any aspect or embodiment of the invention where the amount of SORCS3, or part thereof, or a transcript of the gene encoding same, is present at levels above those of healthy individuals, e.g., above 30% or more, ideally, compared to a control sample or a prior-determined control standard using a group of healthy individuals, or individuals with no history of a panic, concluding that said individual is at an increased risk of developing a disorder including panic or that said test individual is suffering from a disorder including panic.

In preferred embodiment of the invention the amount of SORCS3, or part thereof, is present above 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96% 97%, 98% and 99%, compared to said control.

In a preferred embodiment of the invention the amount of SORCS3, or part thereof, present compared to said control is 41% higher (SORCS3 levels compared to controls). More particularly, in males, the difference was 48% higher and in females 31% higher.

Accordingly, the method may be practiced using a male or female test sample and, in the instance where the test sample is from a male the control amount of SORCS, or part thereof, above which the sample should score to signify panic development or presence, is at least 48%; and in the instance where the test sample is from a female the control amount of SORCS, or part thereof, above which the sample should score to signify panic development or presence, is at least 31%.

According to a further aspect of the invention there is provided a method for diagnosing and treating a subject for the existence of, or susceptibility to, a disorder including panic comprising:
i) performing a method according to the invention to determine the existence of, or susceptibility to developing, panic;
ii) determining a treatment regimen suitable for the treatment of said panic; and
iii) administering one or more panic therapeutics to the subject; and optionally
iv) repeating step i) to determine whether the subject is responding to the treatment and altering the treatment regimen accordingly.

According to yet a further aspect of the invention there is provided a treatment regimen comprising performing said afore method according to any aspect or embodiment of the invention, and then administering an appropriate therapeutic, such as for example a drug, psychosocial intervention or brain stimulation-based treatment or performing an appropriate treatment step. Accordingly, in certain embodiments of the invention SORCS3/SORCS3 represent therapeutic targets for the treatment of panic, in particular an antagonist or agonist against/for same.

According to a further aspect of the invention there is provided an antibody specific for SORCS3, or a part thereof, in particular, the extracellular domain thereof, ideally as shown in FIG. 5, SEQ ID NO: 2.

According to a further aspect of the invention there is provided an antibody specific for SORCS3, or a part thereof, for use in the method of the invention. Preferably, said antibody is specific for the extracellular domain of SORCS3 [SEQ ID NO:2] or a part thereof.

According to a further aspect of the invention there is provided a probe comprising:
i) a sequence which is complementary to at least a part of the sequence structure of SORCS3 shown in FIG. 5; and
ii) a detectable label.

According to yet a further aspect of the invention there is provided a probe comprising:
i) a sequence which is complementary to at least a part of a transcript of the gene encoding SORCS3; and
ii) a detectable label.

Preferably, said probe is labelled to emit a detectable signal upon binding to its target. The nature of the label used will be well known to those skilled in the art and may comprise a radio-label, a light emitting label such as a bioluminescent-labelled or a chemiluminescent-labelled probe or a colour changing label. Alternatively, mass spectrometry may be used to detect binding of target to said probe.

According to a further aspect of the invention there is provided a kit for diagnosing the existence of, or susceptibility to developing panic comprising:
i. at least one antibody specific for SORCS3, or a part thereof; and/or
ii at least one probe specific for SORCS3, or a part thereof, or at least a part of a transcript of the gene encoding SORCS3; and
iii. optionally, reagents and instructions pertaining to the use of said antibody and/or probe.

In a preferred embodiment of the invention said kit further comprises secondary antibodies and/or probes adapted to detect antibodies and/or probes used in the method according to the invention.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with reference to the following wherein:

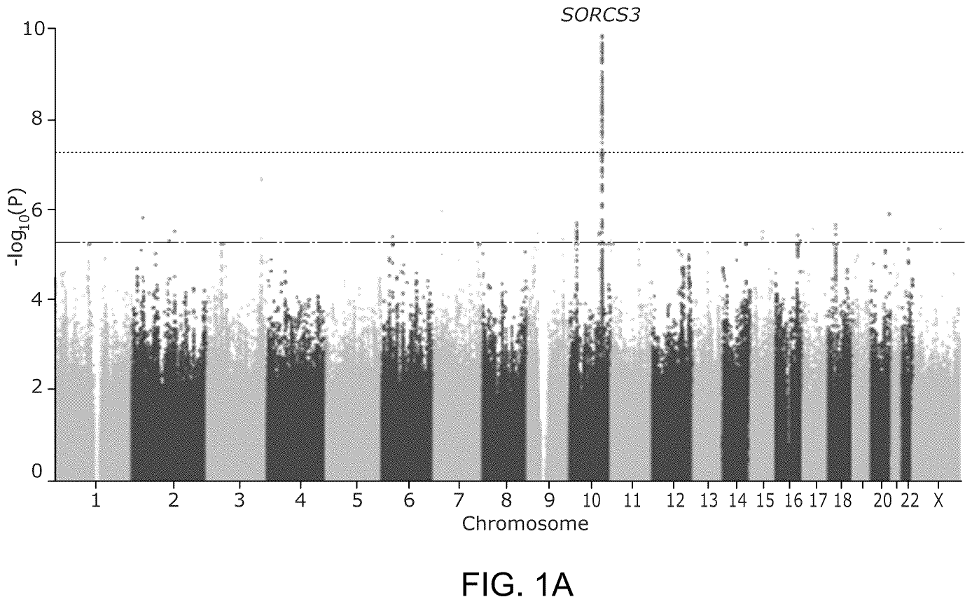

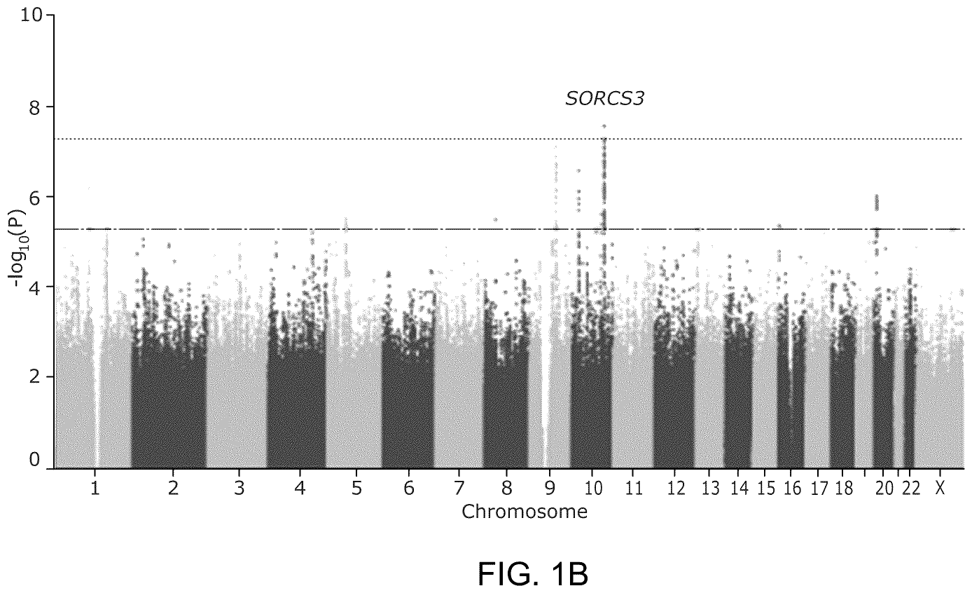

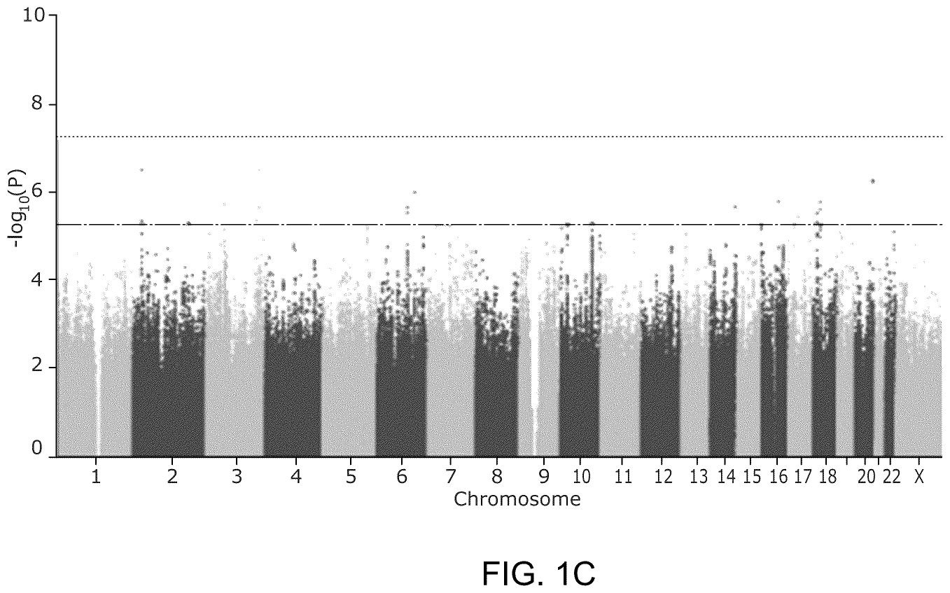

FIG. 1 shows Manhattan plots of GWAS from combined and sex-stratified analyses for Panic Disorder. Genome-wide thresholds for significant (p-value=5.0×10-$) and suggestive (p-value=5.0×10⁻⁶) associations are indicated by the horizontal red and blue lines, respectively. (A).

A novel locus within SORSC3 on chromosome 10q25.1 was significantly associated with Panic Disorder in all subjects from the FinnGen. Sex-stratified analysis showed significant association with the SORCS3 locus in males (B), but not in females (C).

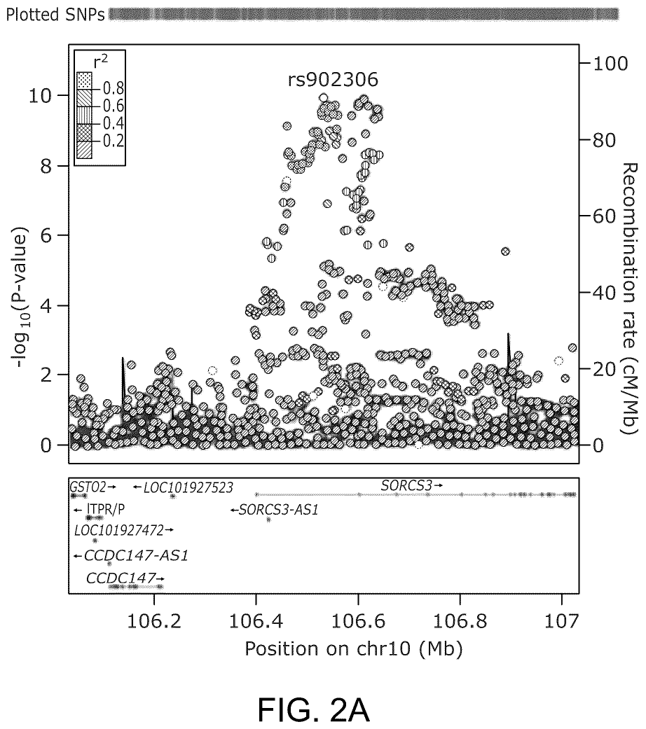

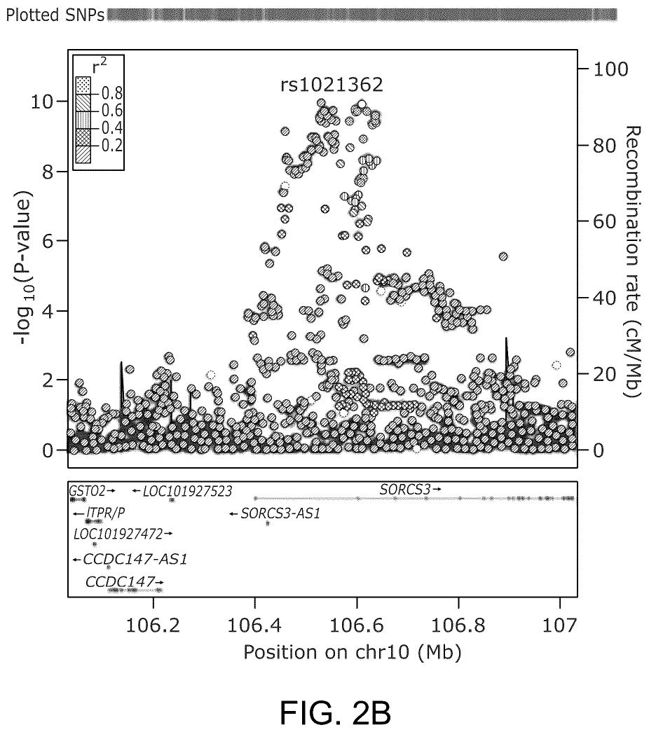

FIG. 2 shows regional plots of SORCS3 locus for Panic Disorder. Each region is centered on the lead SNP (purple diamond) and the genes in the interval are indicated in the bottom panel. The degree of linkage disequilibrium between the lead SNP and other variants is shown as r2 values according to the color-coded legend in the box. Regional plots with (A) GWAS lead SNP rs902306, and (B) an independent significant variant rs1021362 as index SNPs are shown.

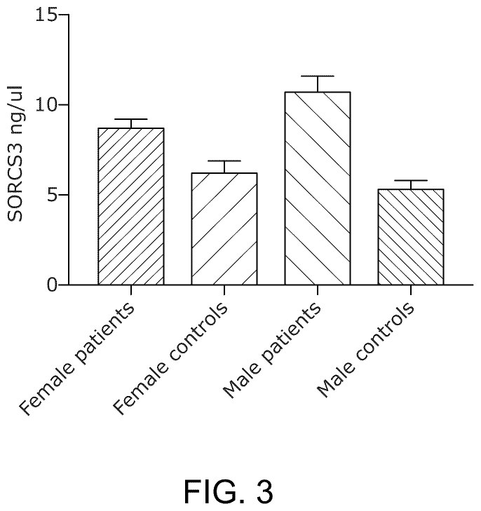

FIG. 3 shows higher serum SORCS3 concentrations in Panic Disorder patients compared to controls. We measured SORCS3 protein concentration by ELISA from 107 patients (52 males and 55 females) and 99 controls (51 males and 48 females). Unadjusted SORCS3 levels are shown for males and females separately. P-values are obtained from a linear regression analysis of log-transformed SORCS3 levels after adjustment for age, sex, analysis batch, and the project id.

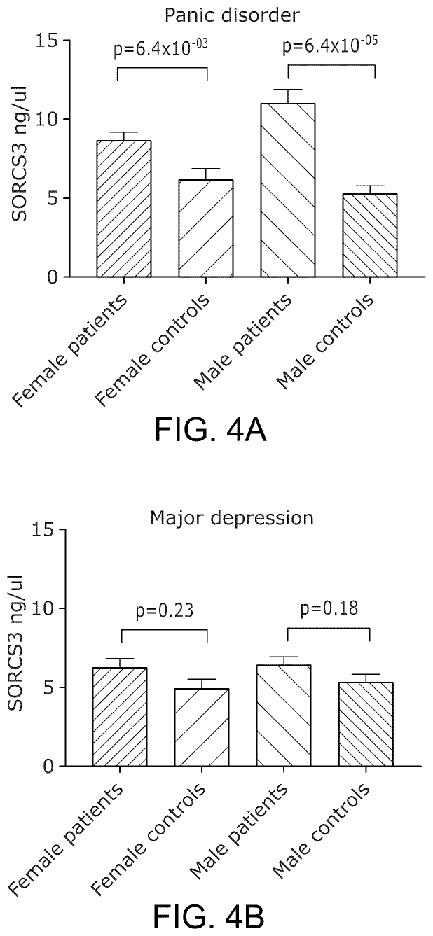

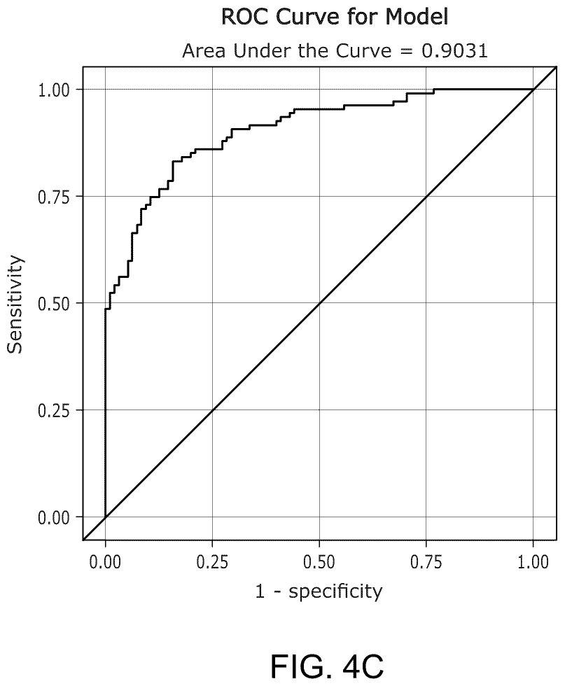

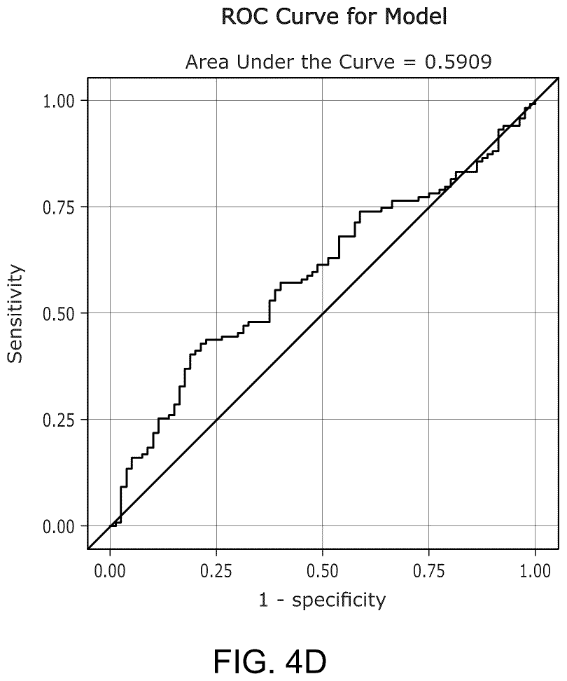

FIG. 4 shows higher serum SORCS3 concentrations in A Panic Disorder patients compared to patients presenting with B Major depression. We measured SORCS3 protein concentration by ELISA from A 107 patients (52 males and 55 females) and 99 controls (51 males and 48 females) and B from 120 MDD patients (58 males and 62 females) and 80 controls (40 males and 40 females). Unadjusted SORCS3 levels are shown for males and females separately. ROC curves for patients presenting with C Panic Disorder patients & D Major depression patients are shown. P-values are obtained from a linear regression analysis of log-transformed SORCS3 levels after adjustment for age, sex, analysis batch, and the project id. (if applicable). Area under the curve for panic disorder (C) and MDD (D) from ROC analysis adjusting for age, sex, analysis batch, and the project id (if applicable) is shown.

FIG. 5 shows the amino acid sequence of SORCS3 comprising SEQ ID NO: 1 (full length protein) or SEQ ID NO: 2 (protein extracellular domain). The extracellular domain is marked by underscoring.

Table 1 shows the clinical characteristics of the FinnGen Study.

Table 2 shows SORCS3 Lead SNP Associations for Panic Disorder in the FinnGen Study.

### Materials and Methods

### FinnGen Study Sample

The FinnGen (https://www.finngen.fi/en) study combines genotype data with longitudinal health register data of Finland, including the causes of death, inpatient, outpatient, and drug reimbursement registers. Panic Disorder cases were defined according to ICD-10 F41.0 or ICD-9 3000B, classified to mild, moderate, or severe Panic Disorder (F41.08, F41.00, F41.01), or unspecified Panic Disorder (F41.09) (Table 1). We excluded cases with psychotic disorders, autism, or intellectual disability. Exclusion criteria for controls included history of any psychiatric endpoint. Additionally, we adjusted the age range of controls to match to that of cases. Based on this approach, we included 3,549 cases and 159,869 control subjects in this study.

### GWAS for Panic Disorder

Subjects from the FinnGen were genotyped with Illumina and Affymetrix arrays (Illumina Inc., San Diego, CA, USA and Thermo Fisher Scientific, Santa Clara, CA, USA).

Genotyping and imputation with the Finnish population-specific SISu v3 reference panel were conducted by the FinnGen.

Briefly, ~16 million single nucleotide polymorphisms (SNPs) were available. After including SNPs with minor allele frequency (MAF) 20.01 that exceeded the quality control criteria (Info score>0.8), 9,362,465 genetic variants spanning autosomal and X chromosomes were available for GWAS in 163,418 individuals from the FinnGen. GWAS was performed using the Scalable and Accurate

Implementation of Generalised mixed model (SAIGE) v0.20¹⁷ with a kinship matrix as a random effect and age, sex, the first 10 principal components (PCs), and genotyping batch as fixed effects.

Additionally, we performed sex-stratified GWAS for Panic Disorder separately for males and females with kinship matrix as a random effect and age, PC1-10, and genotyping batch as fixed effects. Testing equality of coefficients for Panic Disorder from sex-stratified analysis was assessed with a Cochran's Q-test. The dataset uses genome build 38 (hg38).

### Prioritising Variants at the SORCS3 Locus

To functionally validate and prioritise variants at the SORCS3 locus, we first used the Functional Mapping and Annotation of Genome-wide Association Studies (FUMA GWAS) pipeline¹⁸.

Independent SNPs from our GWAS summary results were identified as variants with a p-value<5.0×10⁻⁰⁸ and moderately low linkage disequilibrium (LD) with each other (r2<0.2). As part of FUMA, we also performed gene set analysis using the MAGMA tool¹⁹.

Comparison of Association Signals at the SORCS3 Locus with Panic Disorder and Related Psychiatric Outcomes

The SORCS3 locus has previously been associated with other psychiatric conditions. To further validate that the association signals with our lead SNPs were deriving from Panic Disorder, we performed comparative association analyses with our lead SORCS3 variants (rs902306 and rs1021362) and other psychiatric comorbidities, such as GAD and phobias, and major depressive disorder (MDD). Controls for all comparative analyses remained the same as used in the GWAS for Panic Disorder. First, we tested SORCS3 SNP associations with strict Panic Disorder excluding all cases with comorbid psychiatric endpoints, such as other anxiety disorders, MDD, or substance use disorder. To examine whether SORCS3 association for Panic Disorder may arise due to an overlapping diagnosis with other comorbidities, we next performed analysis for MDD or GAD/phobia with our lead variants where Panic Disorder was excluded from the case definition.

All analyses were performed using SAIGE v0.20¹⁷ with a kinship matrix as a random effect and age, sex, first 10 PCs, and genotyping batch as fixed effects in the model.

### Replication Analyses in the UK Biobank and Estonian Biobank

To investigate whether the association of our SORCS3 lead SNPs replicates in other studies, we acquired summary level data for Panic Disorder with SORCS3 lead variants (rs902306 and rs1021362) from the UK Biobank and the Estonian Biobank.

### Max Planck Institute of Psychiatry Study Sample

Panic Disorder patients were recruited in the Anxiety Disorders Outpatient Unit at the Max Planck Institute of Psychiatry (MPIP) in Munich. DSM-IV Panic Disorder with and without agoraphobia was the primary diagnosis. Panic Disorder due to a medical or neurological condition or the presence of a comorbid Axis II disorder was an exclusion criterion. Control subjects were recruited from a Munich-based community sample and screened for the absence of axis I psychiatric disorders. The analysed sample consisted of 107 patients (52 males and 55 females) and 99 controls (51 males and 48 females).

Evaluation of serum SORCS3 Levels in Panic Disorder Patients and Controls

Serum SORCS3 levels were measured from the MPIP cases and controls with the Human VPS10 Domain-Containing Receptor SORCS3 (SORCS3) ELISA Kit (MyBiosource, San Diego, USA) according to manufacturer's instructions. Two samples were below the detection limit of the kit (0.5 ng/ml) and were excluded from the analyses. The analysis was carried out in two batches balanced by sex, with 54 patients and 69 controls, and 53 patients and 30 controls. Obtained SORCS3 concentration values were log-transformed, followed by combined and sex-stratified linear regression analysis with Panic Disorder as an exposure variable, after adjustment for age, sex (if applicable), batch, and project. Lastly, an interaction term between Panic Disorder and sex was added to the model to test whether SORCS3 levels by patient status differ by sex.

### Results

### Identification of SORCS3 as a Novel Locus for Panic Disorder

We identified 132 genome-wide significant (p-value<5.0×10⁻⁰⁸) SNPs through a GWAS for Panic Disorder in the FinnGen (FIG. 1A). All variants localised in chr10q25.1 in sortilin related VPS10 domain containing receptor 3 (SORCS3) locus (FIG. 1A). Minor allele T of our lead variant rs902306 was associated with a 22% increased risk of Panic Disorder among 3,549 cases and 159,869 controls free from any psychiatric end points (OR=1.22, 95% CI 1.15-1.30, p-value 1.1×10⁻¹⁰) (Table 2).

Although all our significant GWAS associations for Panic Disorder were localised within SORCS3, we utilised FUMA GWAS pipeline¹⁸ to identify additional signals in this locus. This analysis yielded one independent significant SNP, rs1021362, where minor G-allele (MAF 27%) was associated with 20% increased risk of Panic Disorder (OR=1.20, 95% CI 1.13-1.27, p-11 value=1.2×10⁻¹⁰) (Table 2). Rs1021362 is located 79 kb downstream from rs902306 and is in moderately low linkage disequilibrium (LD) with our lead SNP (r2=0.29 in Finns, r2=0.19 in all Europeans). As part of FUMA GWAS18, we performed a gene set analysis using MAGMA20, which did not reveal additional genes associated with Panic Disorder. Moreover, regional plot of the SORCS3 locus showed two distinct LD clusters, with rs902306 and rs1021362 as the most significant SNPs of these nearby regions (FIG. 2A-B).

We also performed sex-stratified GWAS for Panic Disorder, but these analyses did not reveal any additional loci for Panic Disorder (FIG. 1B-C). The association with rs902306 was genome-wide significant only in males (OR=1.35, 95% CI 1.21-1.51, p-value=2.3×10⁻⁰⁷), but not in females (OR=1.17, 95% CI 1.09-1.26, p-value 9.8×10⁻⁰⁶; p-value for heterogeneity=0.04)(Table 2). Comparable sex-stratified results were detected with rs1021362 (OR=1.32, 95% CI 1.18-1.46, p-value=1.9×10⁻⁰⁷ in males; and OR=1.16, 95% CI 1.09-1.12, p-value 9.5×10⁻⁶ in females; p-value for heterogeneity=0.04) (Table 2). However, p-values for heterogeneity did not remain significant after Bonferroni correction for testing 2 SNPs (p<0.05/2=0.025), suggesting no significant sex differences. GWAS for Panic Disorder in males only showed genome-wide significant association with one SNP (rs1490176, OR=1.36, 95% CI 1.22-1.52, p-value=2.6×10⁻⁰⁸), which also localised in SORCS3 (~28 kb from rs902306) and was in moderately high linkage disequilibrium with our lead SNP rs902306 (r²=0.79 in Finns, r²=0.87 in other Europeans). This variant has been linked to various psychiatric endpoints previously. In the combined analyses (males and females), rs1490176 was also significantly associated with Panic Disorder (OR=1.20, 95% CI 1.13-1.28, p-value=3.5×10⁻¹⁰), comparable with our lead SNP. GWAS in females only did not yield genome-wide significant findings.

Lastly, we performed a phenome-wide association study (PheWAS) with our SORCS3 variants. We utilised FUMA GWAS¹³ to identify genome-wide significant (p-value<5.0×10⁻⁰⁸) associations with our SORCS3 variants and/or proxy SNPs with high LD (r²>0.8) with our lead SNPs and other phenotypes. Our lead SNPs have not been significantly associated with any other traits in previous GWAS studies. However, the chr10q25.1 locus has been associated with various traits and psychiatric conditions previously, including major depressive disorder (MDD).

Comparative Analyses with Other Psychiatric Disorders

Panic Disorder is often comorbid with other psychiatric disorders. To examine whether our SORCS3 association can be attributed to Panic Disorder, we performed comparative analyses in the FinnGen with different Panic Disorder comorbidities. First, we performed a SNP association with lead SORCS3 variants for Panic Disorder after excluding all other psychiatric comorbidities from case-definition (Panic Disorder only). In this analysis, SORCS3 variants increased the disease risk by ~22% (OR=1.20, 95% CI 1.04-1.37, p-value=0.01; OR=1.24, 95% CI 1.10-1.41, p-value=6.3×10⁻⁰⁴ with rs902306 and rs1021362, respectively). Effect estimates for Panic Disorder remained virtually unchanged compared to those without excluded psychiatric comorbidities, but significance was mitigated most likely due to substantial decrease in the number of cases (n=628) (Table 2).

Next, we tested whether SORCS3 variants were associated with general anxiety disorder (GAD) or phobias after exclusion of Panic Disorder from the case definition. There was a modest association with rs902306 and risk of GAD/phobia (OR=1.07, 95% CI 1.01-1.13, p-value=0.03), but not with rs1021362. Sample size for GAD/phobia (3,738 cases and 161,438 controls) was similar as for Panic Disorder used in our discovery GWAS (Table 2).

Lastly, we tested whether SORCS3 variants were associated with MDD. After exclusion of Panic Disorder diagnosis from the case definition, rs902306 T-allele was associated with 5% increased risk for MDD in 25,906 cases and 161,371 controls (OR=1.05, 95% CI 1.03-1.08, p-value 6.4×10⁻⁰⁵). Similarly, rs1021362 G-allele was increasing the risk of MDD by 5% (OR=1.05, 95% CI 1.02-1.07, p-value=6.6×10⁻⁰⁵). Although our SORCS3 variants showed modest association with MDD, the effect estimates and significance was weaker for MDD compared to Panic Disorder, despite of much larger sample size (Table 2).

### Replication Analyses in the UK Biobank and the Estonian Biobank

To investigate whether our SORCS3 lead SNPs rs902306 and rs1021362 associate with Panic Disorder in other cohorts, we acquired summary level data from the UK Biobank and the Estonian Biobank. Neither of the SNPs associated with Panic Disorder in these samples.

### Higher Serum SORCS3 Levels in Panic Disorder Cases Compared to Controls

The extracellular domain of the SORCS3 protein can be shed through a regulated proteolytic cleavage and detected in the blood. To investigate whether SORCS3 protein concentrations differ between Panic Disorder cases and controls, we carried out ELISA using serum from an independent sample of 107 German Panic Disorder patients and 99 controls. The mean SORCS3 levels were 10.14 ng/ml in male patients, 5.22 ng/ml in male controls, 8.54 ng/ml in female patients, and 5.82 ng/ml in female controls. Overall, Panic Disorder patients had 41% higher SORCS3 serum levels compared to controls (B=0.703, SE=0.151, p-value=3.4×10⁻⁰⁶) In males, the difference was 48.5% (beta=0.868, SE=0.225, p-value=1.2×10⁻⁰⁴) and in females 31.9% (B=0.548, SE=0.206, p-value=9.1×10⁻⁰³) (FIG. 3). SORCS3 levels by Panic Disorder were not significantly different between males and females (p-interaction=0.31).

### Higher Serum SORCS3 Levels in Panic Disorder Cases Compared to Major Depression Cases

As above we used the shed extracellular domain of the SORCS3 protein in blood to investigate whether SORCS3 protein concentrations differs between Panic Disorder cases and Major Depression cases (FIG. 4).

We measured the serum SORCS3 levels from 120 patients with major depressive disorder (MDD) and 80 controls, collected from the Max Planck Institute for Psychiatry. Obtained SORCS3 concentration values were log-transformed, followed by combined and sex-stratified linear regression analysis with MDD as an exposure variable, after adjustment for age and sex.

The mean SORCS3 levels were 10.14 ng/ml in male patients, 5.22 ng/ml in male controls, 8.54 ng/ml in female patients, and 5.82 ng/ml in female controls. Overall, Panic Disorder patients had a significantly higher SORCS3 serum levels compared to controls (p=6.410⁻⁰³, for females and p-value=6.4×10⁻⁰⁵ for males) FIG. 4A.

In contrast, male and female patients presenting with Major Depression did show a significant difference in serum SORCS3 levels compared with controls, FIG. 4B.

To evaluate the performance of our binary classification model and whether SORCS3 levels can be utilized to diagnose patients with and without the disease, we constructed a receiver operating characteristic (ROC) curve for panic disorder using logistic regression and plotting predicted probabilities after including serum SORCS3 levels, age, sex, batch, and project in the model. In comparison, we also constructed a ROC curve for MDD after fitting SORCS3 levels, age, and sex in the model.

### CONCLUSIONS

To identify genetic loci associated with Panic Disorder, we carried out a GWAS in the Finnish register-based FinnGen study. We identified significant association with SNPs in the intronic regions of SORCS3. We also found higher serum SORCS3 protein levels in Panic Disorder (with and without agoraphobia) patients compared to controls. Although SORCS3 locus has been linked to other psychiatric endpoints previously, this is the first report of SORCS3 associating to Panic Disorder.

It is unlikely that the SORCS3 association with Panic Disorder is specific for the Finnish population because the risk allele frequency of rs902306 is similar in the FinnGen subjects (21%) and other European populations (20-27%).

SORCS3 belongs to the VPS10P domain gene family, with suggested functions in brain plasticity and behaviour. Like other VPS10P domain family members, SORCS3 protein's extracellular domain can be shed by an enzymatic cleavage and the cleaved protein may function as a carrier for its ligands, such as the platelet-derived growth factor BB, nerve growth factor, or tropomyosin-related kinase B. We found 41% higher SORCS3 serum levels in German Panic Disorder patients compared to controls. SORCS3 serum levels have not been previously associated with clinical phenotypes. SORCS3 expression is highest in the brain, but lower expression levels are also detectable in the adrenal glands and peripheral nerves according to the Genotype-Tissue Expression Project. Our data suggest SORCS3 as a serum biomarker for panic e.g., Panic Disorder. Moreover, SORCS3 is a specific marker correlating with Panic Disorder but not other neurological disorders such as depression.

Revealing the neurobiological mechanisms by which SORCS3 regulates anxiety will help understand the etiology of panic in a number of psychiatric and psychiatric-related disorders.

TABLE 1
Clinical characteristics of the FinnGen Study.

| | All | Females | Males |
|---|---|---|---|
| Panic disorder | | | |
| Cases | 3 549 | 2 592 | 957 |
| Controls | 159 869 | 86 937 | 72 932 |
| Mean year of birth | | | |
| Cases | 1970 (16.8) | 1972 (16.8) | 1967 (16.3) |
| Controls | 1959 (17.1) | 1961 (17.1) | 1956 (16.7) |
| ^{a}Panic disorder diagnoses | | | |
| Panic disorder (F41.0) | 3 467 | 2 550 | 917 |
| ^{b}Mild panic disorder (F41.08) | 151 | 118 | 33 |
| ^{b}Moderate panic disorder | 1 046 | 771 | 275 |
| (F41.00) | | | |
| ^{b}Severe panic disorder | 272 | 198 | 74 |
| (F41.01) | | | |
| Unspecified panic disorder | 1 854 | 1 371 | 483 |
| (F41.09) | | | |
| Panic disorder (3000B) | 85 | 46 | 39 |
| Broad definition of panic | 31 | 11 | 20 |
| disorder (3002B) | | | |
| Psychiatric comorbidities | | | |
| among cases | | | |
| No psychiatric comorbidities | 628 | 434 | 194 |
| ^{c}Major depressive disorder | 2 023 | 1 530 | 493 |
| (MDD) | | | |
| ^{d}Generalised anxiety disorder | 524 | 402 | 122 |
| (GAD) | | | |
| ^{e}Other anxiety or stress | 2 208 | 1 671 | 537 |
| disorder | | | |

Data is shown as number of subjects or year of birth (SD).
^{a}Panic disorder cases were defined as follows: ICD-10 F41.0, F41.08, F41.00, F41.01, F41.09; ICD-9 3000B, 3002B. Control subjects were free from any psychiatric diagnosis.
^{b}Severity of panic disorder was defined as mild: <4 panic attacks within a month; moderate: ≥4 panic attacks within a month; severe: ≥4 panic attacks within a week.
^{c}MDD cases were defined as follows: ICD-10 F32, F33; ICD-9 2961; ICD-8 2980, 3004, 79020.
^{d}GAD cases were defined as follows: ICD-10 F41.1; ICD-9 3000C.
^{e}Other anxiety or stress disorder cases were defined as follows: ICD-10 F40.00, F40.01, F40.1, F40.2, F40.8, F40.09, F41.1, F41.2, F41.3, F41.8, F41.9, F42, F43, F44, F45, F48; ICD-9 3000A, 3000C, 3001, 3003, 3004, 3006, 3007, 3008; ICD-8 30000, 3001, 3003, 3004, 3005, 3006, 3007, 3008, 3009.

TABLE 2
SORCS3 Lead SNP Associations for Panic Disorder in the FinnGen Study.

| | | | ^{a}rs902306 | | ^{a}rs1021362 | |
|---|---|---|---|---|---|---|
| Position (hg38) | | | chr10: 104,772,457 | | chr10: 104,851,510 | |
| EA/NEA | | | T/C | | G/A | |
| EAF | | | 0.21 | | 0.27 | |
| Strata | Cases | Controls | OR (95% CI) | p-value | OR (95% CI) | p-value |
| All | 3 549 | 159 869 | 1.22 (1.15-1.30) | 1.1 × 10⁻¹⁰ | 1.20 (1.13-1.27) | 1.2 × 10⁻¹⁰ |
| Male | 957 | 72 932 | 1.35 (1.21-1.51) | 2.3 × 10⁻⁰⁷ | 1.32 (1.18-1.46) | 1.9 × 10⁻⁰⁷ |
| Female | 2 592 | 86 937 | 1.17 (1.09-1.26) | 9.8 × 10⁻⁰⁶ | 1.16 (1.09-1.23) | 9.5 × 10⁻⁰⁶ |

EA, effect allele;
NEA, non-effect allele,
EAF, effect allele frequency;
OR, odds ratio;
CI, confidence interval.
^{a}Effect estimates were stronger in males compared to females with both variants (heterogeneity p-value = 0.04)

### REFERENCES

12. Otowa T, Yoshida E, Sugaya N, et al. Genome-wide association study of panic disorder in the Japanese population. J Hum Genet. February 2009; 54(2):122-6. doi:10.1038/jhg.2008.17
13. Erhardt A, Czibere L, Roeske D, et al. TMEM132D, a new candidate for anxiety phenotypes: evidence from human and mouse studies. Molecular psychiatry. 2011; 16(6):647-663.
14. Erhardt A, Akula N, Schumacher J, et al. Replication and meta-analysis of TMEM132D gene variants in panic disorder. Translational psychiatry. 2012; 2(9):e156-e156.
15. Forstner A J, Awasthi S, Wolf C, et al. Genome-wide association study of panic disorder reveals genetic overlap with neuroticism and depression. Mol Psychiatry. August 2021; 26(8):4179-4190. doi:10.1038/s41380-019-0590-2
17. Zhou W, Nielsen J B, Fritsche L G, et al. Efficiently controlling for case-control imbalance and sample relatedness in large-scale genetic association studies. Nat Genet. September 2018; 50(9):1335-1341. doi:10.1038/s41588-018-0184-y
18. Watanabe K, Taskesen E, van Bochoven A, Posthuma D. Functional mapping and annotation of genetic associations with FUMA. Nat Commun. Nov. 28, 2017; 8(1):1826. doi:10.1038/s41467-017-01261-5
19. de Leeuw C A, Mooij J M, Heskes T, Posthuma D. MAGMA: generalized gene-set analysis of GWAS data. PLoS Comput Biol. April 2015; 11(4):e1004219. doi:10.1371/journal.pcbi.1004219

1. A method for determining the existence of, or susceptibility to developing, panic in an individual to be tested comprising:
(a) providing a sample from said individual to be tested;
(b) detecting, or quantifying the amount of SORCS3 protein/polypeptide, or a part thereof, or a transcript of a gene encoding same, in said sample; and
(c) when the amount of SORCS3, or part thereof, or a transcript of the gene encoding same, is present at a level above that in healthy individuals, or in a control sample, or in a prior determined control standard using a group of healthy individuals, or in individuals with no history of panic or that said test individual is suffering from a panic disorder.

2. The method according to claim 1, wherein said sample is selected from the group consisting of: serum, urine, seminal fluid, blood, blood plasma, lymph fluid, CSF, saliva, sputum, lavage, bronchoalveolar lavage, cerebrospinal fluid and extracellular vesicles.

3. (canceled)

4. The method according to claim 1, wherein said method is undertaken in respect of a psychiatric disorder or a psychiatric-related disorder selected form the group consisting of: generalised anxiety disorder (GAD), obsessive-compulsive disorder (OCD), panic disorder (PD), PD with agoraphobia, post-traumatic stress disorder (PTSD), social anxiety disorder (also called social phobia), specific phobias, panic associated within other psychiatric disorders, panic arising from a primary medical condition, such as COPD and heart disease.

5. The method according to claim 1, wherein said part of SORCS3 is the extracellular domain of the protein.

6. (canceled)

7. The method according to claim 1, wherein said transcript is mRNA.

8. (canceled)

9. The method according to claim 1, wherein said method is an immunoassay, an enzyme-based assay, an enzyme-linked immunosorbent assay (ELISA), or an immunofluorescence-based assay.

10-12. (canceled)

13. The method according to claim 1, wherein said sample is contacted with one or more probes wherein one or more of said probe is specific for, and so detects, SORCS3, or a part thereof, or a transcript of the gene encoding same, in said sample.

14. The method according to claim 13, wherein said probe is provided with a signalling agent that emits a signal when the probe is bound to its target and so wherein said signal is indicative of the presence or amount of said SORCS3, or a part thereof, or a transcript of the gene encoding same, in said sample.

15. The method according to claim 1, wherein said method involves contacting said sample with one or more further probe wherein said further probe detects SORCS3, or a part thereof, or a transcript of the gene encoding same, and/or an earlier used probe.

16. The method of claim 1, wherein SORCS3 is human.

17. The method according to claim 1, wherein SORCS3 is characterised by SEQ ID NO: 1 and/or SEQ ID NO: 2.

18. The method according to claim 17, wherein either one, or both, of SEQ ID NO:1 or SEQ ID NO:2 is/are detected in said method.

19. The method according to claim 1, wherein:
said detecting or quantifying is undertaken by assessing the binding of an antibody to SORCS3, or a part thereof; or
said detecting or quantifying is undertaken by assessing the binding of a probe to SORCS3, or a part thereof, or a transcript of the gene encoding same.

20-21. (canceled)

22. The method according to claim 1, wherein the amount of SORCS3, or part thereof, or a transcript of the gene encoding same, is present above a % selected from the group consisting of: 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96% 97%, 98% and 99%, compared to healthy individuals, or in a control sample, or in a prior determined control standard using a group of healthy individuals, or in individuals with no history of panic.

23. The method according to claim 22 wherein the amount of SORCS3, or part thereof, or a transcript of the gene encoding same, present compared to said control is at least 41% higher.

24. The method according to claim 22 wherein the amount of SORCS3, or part thereof, or a transcript of the gene encoding same, present compared to said control is 48% higher in males and/or 31% higher in females.

25. A method for diagnosing and treating a subject for the existence of, or susceptibility to develop panic comprising:
i) performing a method according to claim 1 to determine the existence of, or susceptibility to develop panic;
ii) determining a treatment regimen suitable for the treatment of said panic; and
iii) administering one or more panic therapeutics or treatments to the subject; and optionally
iv) repeating step i) to determine whether the subject is responding to the treatment and altering the treatment regimen accordingly.

26. A treatment regimen comprising performing said method according to claim 1, and then administering an appropriate therapeutic or performing an appropriate treatment step.

27-28. (canceled)

29. A probe comprising:
i) a sequence which is complementary to at least a part of the sequence structure of SORCS3, or at least part of a transcript of the gene encoding SORCS3, wherein the SORCS3 amino acid sequence comprises SEQ ID NO: 1 and/or SEQ ID NO: 2; and
ii) a detectable label.

30. (canceled)

31. A kit for diagnosing the existence of, or susceptibility to developing panic comprising:
i. at least one antibody specific for SORCS3, or a part thereof, wherein the amino acid sequence of SORCS3 comprises SEQ ID NO: 1 and/or SEQ ID NO: 2; and/or
ii. at least one probe specific for SORCS3, or a part thereof, or at least a part of a transcript of the gene encoding SORCS3, wherein the amino acid sequence of SORCS3 comprises SEQ ID NO: 1 and/or SEQ ID NO: 2; and
iii. optionally, reagents and instructions pertaining to the use of said antibody and/or probe.


SEQUENCE LISTING (2 sequences)

SEQ ID NO: 1 - protein, 1222 aa, Homo sapiens
MEAARTERPAGRPGAPLVRTGLLLLSTWVLAGAEITWDATGGPGRPAAPASRPPALSPLSPRAVASQWPEELASARRAAVLGRRAGPELLPQQGGGRGGEMQVEAGGTSPAGERRGRGIPAPAKLGGARRSRRAQPPITQERGDAWATAPADGSRGSRPLAKGSREEVKAPRAGGSAAEDLRLPSTSFALTGDSAHNQAMVHWSGHNSSVILILTKLYDFNLGSVTESSLWRSTDYGTTYEKLNDKVGLKTVLSYLYVNPTNKRKIMLLSDPEMESSILISSDEGATYQKYRLTFYIQSLLFHPKQEDWVLAYSLDQKLYSSMDFGRRWQLMHERITPNRFYWSVAGLDKEADLVHMEVRTTDGYAHYLTCRIQECAETTRSGPFARSIDISSLVVQDEYIFIQVTTSGRASYYVSYRREAFAQIKLPKYSLPKDMHIISTDENQVFAAVQEWNQNDTYNLYISDTRGIYFTLAMENIKSSRGLMGNIIIELYEVAGIKGIFLANKKVDDQVKTYITYNKGRDWRLLQAPDVDLRGSPVHCLLPFCSLHLHLQLSENPYSSGRISSKETAPGLVVATGNIGPELSYTDIGVFISSDGGNTWRQIFDEEYNVWFLDWGGALVAMKHTPLPVRHLWVSFDEGHSWDKYGFTSVPLFVDGALVEAGMETHIMTVFGHFSLRSEWQLVKVDYKSIFSRHCTKEDYQTWHLLNQGEPCVMGERKIFKKRKPGAQCALGRDHSGSVVSEPCVCANWDFECDYGYERHGESQCVPAFWYNPASPSKDCSLGQSYLNSTGYRRIVSNNCTDGLREKYTAKAQMCPGKAPRGLHVVTTDGRLVAEQGHNATFIILMEEGDLQRTNIQLDFGDGIAVSYANFSPIEDGIKHVYKSAGIFQVTAYAENNLGSDTAVLFLHVVCPVEHVHLRVPFVAIRNKEVNISAVVWPSQLGTLTYFWWFGNSTKPLITLDSSISFTFLAEGTDTITVQVAAGNALIQDTKEIAVHEYFQSQLLSFSPNLDYHNPDIPEWRKDIGNVIKRALVKVTSVPEDQILIAVFPGLPTSAELFILPPKNLTERRKGNEGDLEQIVETLFNALNQNLVQFELKPGVQVIVYVTQLTLAPLVDSSAGHSSSAMLMLLSVVFVGLAVFLIYKFKRKIPWINIYAQVQHDKEQEMIGSVSQSENAPKITLSDFTEPEELLDKELDTRVIGGIATIANSESTKEIPNCTSV

SEQ ID NO: 2 - protein, 1092 aa, Homo sapiens
EITWDATGGPGRPAAPASRPPALSPLSPRAVASQWPEELASARRAAVLGRRAGPELLPQQGGGRGGEMQVEAGGTSPAGERRGRGIPAPAKLGGARRSRRAQPPITQERGDAWATAPADGSRGSRPLAKGSREEVKAPRAGGSAAEDLRLPSTSFALTGDSAHNQAMVHWSGHNSSVILILTKLYDFNLGSVTESSLWRSTDYGTTYEKLNDKVGLKTVLSYLYVNPTNKRKIMLLSDPEMESSILISSDEGATYQKYRLTFYIQSLLFHPKQEDWVLAYSLDQKLYSSMDFGRRWQLMHERITPNRFYWSVAGLDKEADLVHMEVRTTDGYAHYLTCRIQECAETTRSGPFARSIDISSLVVQDEYIFIQVTTSGRASYYVSYRREAFAQIKLPKYSLPKDMHIISTDENQVFAAVQEWNQNDTYNLYISDTRGIYFTLAMENIKSSRGLMGNIIIELYEVAGIKGIFLANKKVDDQVKTYITYNKGRDWRLLQAPDVDLRGSPVHCLLPFCSLHLHLQLSENPYSSGRISSKETAPGLVVATGNIGPELSYTDIGVFISSDGGNTWRQIFDEEYNVWFLDWGGALVAMKHTPLPVRHLWVSFDEGHSWDKYGFTSVPLFVDGALVEAGMETHIMTVFGHFSLRSEWQLVKVDYKSIFSRHCTKEDYQTWHLLNQGEPCVMGERKIFKKRKPGAQCALGRDHSGSVVSEPCVCANWDFECDYGYERHGESQCVPAFWYNPASPSKDCSLGQSYLNSTGYRRIVSNNCTDGLREKYTAKAQMCPGKAPRGLHVVTTDGRLVAEQGHNATFIILMEEGDLQRTNIQLDFGDGIAVSYANFSPIEDGIKHVYKSAGIFQVTAYAENNLGSDTAVLFLHVVCPVEHVHLRVPFVAIRNKEVNISAVVWPSQLGTLTYFWWFGNSTKPLITLDSSISFTFLAEGTDTITVQVAAGNALIQDTKEIAVHEYFQSQLLSFSPNLDYHNPDIPEWRKDIGNVIKRALVKVTSVPEDQILIAVFPGLPTSAELFILPPKNLTERRKGNEGDLEQIVETLFNALNQNLVQFELKPGVQVIVYVTQLTLAPLVDSSAGHSSS